(19) United States
(12) Patent Application Publication
(10) Pub. No.: US 2006/0252706 A1
(43) Pub. Date: Nov. 9, 2006

(54) Composition for slimming

(76) Inventors: Ji-Hyun Kim, Gyeonggi-do (KR); Soo-Mi Ahn, Gyeonggi-do (KR); Jong-Chan Lee, Gyeonggi-do (KR); Young-Kyung Kim, Gyeonggi-do (KR); Byeong-Gon Lee, Gyeonggi-do (KR); Sun-Young Kim, Gyeonggi-do (KR); Ji-Eun Park, Seoul (KR); Hyung-Woo Park, Gyeonggi-do (KP); Sang-Jun Lee, Gyeonggi-do (KR); Hak-Hee Kang, Gyeonggi-do (KR)

Correspondence Address: SUGHRUE MION, PLLC, 2100 PENNSYLVANIA AVENUE, N.W., SUITE 800, WASHINGTON, DC, 20037, US

(21) Appl. No.: 10/554,050

(22) PCT Filed: Apr. 23, 2004
(86) PCT No.: PCT/KR04/00947

§ 371 Date: Jan. 25, 2006

(30) Foreign Application Priority Data
Apr. 24, 2003 (KR) 1020030026015
Dec. 29, 2003 (KR) 1020030098859

(51) Int. Cl. A61K 31/7048; A61K 31/522; A61K 31/353; A61K 31/22

(52) U.S. Cl. 514/27; 514/263.34; 514/456; 514/546

(57) ABSTRACT

The present invention relates to a composition for slimming, more particularly, to a slimming composition containing theanine and at least on selected from the group consisting of caffeine, genistein, L-carnitine and catechin. The composition of the present invention contains theanine and each of caffeine, genistein, L-carnitine or mixtures thereof, and has properties of decomposing fats, hydrolyzing lipid and removing cellulites.

### FIELD OF THE INVENTION

The present invention relates to a composition for slimming, more particularly, to a slimming composition containing theanine and at least one selected from the group consisting of caffeine, genistein, L-carnitine and catechin. The composition of the present invention contains theanine and each of caffeine, genistein, L-carnitine, catechin or mixtures thereof, and has properties of decomposing fats, hydrolyzing lipid and removing cellulites.

### BACKGROUND OF THE INVENTION

A human body has about 20 billions of fat cells, which store and release energies in the body. There are complex mechanisms of storing and releasing energies in a body, and when the amount of energy supplied is more than that of consumed the energy is stored as neutral fat (lipid) in the fat cells (adipocytes), and when energy is required the fats are hydrolyzed as fatty acid and glucose to be used as energy. Obesity appears when the energy balance is broken in this mechanism and excessive energy is accumulated, and as a result, fat cells become bigger or the number of fat cells increases.

It is reported that about 30˜40% of the moderns have obesity, and because the obesity usually accompanies with geriatric diseases such as hypertension, hyperlipemia, arteriosclerosis, cardiac disorder, diabetes, or the like, it is an important concern to treat obesity. In addition, not only for health but also for beauty, treating obesity and maintaining preferable physical condition and body figure are great concerns. As a viewpoint of beauty, the desire for cosmetics for slimming and anti-cellulite, which is effective to remove excessive subcutaneous fat or to improve firmness or elasticity of skin, also increases.

Cellulite is generated in the skin and subcutaneous fat, and makes the skin rough like a peel of an orange due to the accumulations of fat and waste materials thereof. Even though obesity is not a direct reason of the generation of cellulite, cellulite increases when the size or the number of fat cells become larger, and therefore it is very effective to decompose and remove fats in fat cells in order to maintain preferable body figure and skin state.

Therefore, various methods for treating obesity have been studied in the viewpoints of health or beauty. Conventional methods for treating obesity comprise a diet cure, an exercise cure, a surgical cure or a drug cure in order to reduce intake of energy or to increase consumption of energy. However, because these methods cannot solve the problem of obesity completely and have severe side effects, effectiveness or safety is not guaranteed yet. In addition, in the viewpoint of beauty, because the treatment of obesity should consider the improvement of skin state as well as removing fats, the above methods are not sufficient to be applied. Therefore it is required to find a new material having similar or better effects than those of the conventional materials without side effects.

Considering the causes of obesity and the disease induced therefrom, an important thing is not just reducing body weight but reducing the body fat. Therefore, it is necessary to find a method for increasing the decomposition or combustion of unnecessary fats accumulated in the body.

The present inventors studied and researched to find a safe material that can improve decomposition or hydrolysis of fats and accelerate combustion of fats, and finally found that theanine, caffeine, genistein, L-carnitine and catechin have the properties of hydrolyzing lipid, removing cellulites and decomposing fats effectively and can help maintaining preferable elastic and firm body figure.

### SUMMARY OF THE INVENTION

The present invention provides a novel use of theanine which can improve the decomposition of neutral fats and can accelerate the expression of β₃-adrenergic receptor to help the combustion of fats; a novel use of caffeine which can suppress the expression of phosphodiesterase, an enzyme that inhibits the decomposition of fats in a fat cell, to improve the decomposition of fats; a novel use of genistein which can accelerate the decomposition and combustion of fats; a novel use of L-carnitine mixture which can accelerate the functions of genistein decomposing fats; a novel use of catechin which can suppress the differentiation of fat cells (adipocytes).

The present invention also provides an external composition for slimming containing theanine and at least one of caffeine, genistein, L-carnitine and catechin, which has the properties of decomposing fats, hydrolyzing lipid, removing cellulites and reducing roughness on the skin induced by the cellulite to enhance or recover the firmness and the elasticity of the skin.

In addition, the present invention provides a method for removing body fat by applying the above slimming composition on the skin to decompose fats.

### DETAILED DESCRIPTION OF THE INVENTION

In order to accomplish the object of the invention, the composition for slimming of the present invention comprises theanine and at least one selected from the group consisting of caffeine, genistein, L-carnitine and catechin, which enhances the metabolism of the fat cells to improve decomposition of fats.

Hereinafter, the present invention is described in detail.

The term “slimming” means inhibiting and reducing obesity as well as reducing cellulite to make preferable body figure and firm and elastic smooth skin.

Theanine is a kind of amino acid that exhibits the specific taste of green tea, and it is reported that when a person takes theanine, α-ray increases, which makes a person relaxed and stabilized (Nippon Nogeikagaku Kaishi. 72(2), 153-157 (1998)). The theanine of the present invention comprises L-form, which is extracted from green tea, and L-theanine, D-theanine and DL-theanine that are synthesized, and any form of the theanines can be used in this.

Caffeine, known as a positive control of an enhancer for decomposition of fat, is a kind of methylxanthine material that shows a property of decomposing fat, which suppresses the expression of phosphodiesterase—an important material in the decomposition of fat—to increase cAMP in the cell (Astrup, A. et al., Am J. Clin. Nutr. 51: 759, 1990).

Genistein is a kind of isoflavone generally contained in soybeans, and is a vegetable hormone similar to a female hormone having various physiological activities, and it is reported that the genistein has the properties of controlling metabolism of fat in a adipocyte (J. Steroid Biochem Mol Biol. 75(4-5): 265-71 (2000)), and reducing blood cholesterol (J. Nutr. January; 126(1): 43-50 (1996)).

L-carnitine is an essential nutrient that is synthesized in a liver or a kidney and is generally contained in food, especially in red meat. It is reported that when L-carnitine is insufficient, generation of energy decreases because concentration of fatty acid in a mitochondria decreases, and that L-carnitine has various properties of anti-aging effects, reducing blood fat, enhancing heart function, or the like (Robert Crayhon, M.S., Carnitine miracle).

Catechin is an important component of green tea having various medical functions such as anti-oxidation effect, anti-cancer effect or inhibition of heart disease. Various catechins comprising (+) catechin (C), (−) epicatechin (EC), (−) epigallocatechin-3-gallate (EGCG), (+) epigallocatechin (EGC), (−) epicatechin gallate (ECG) are reported up to now. The catechins of the present invention are extracted from green tea and main components thereof comprise EC, EGC, EGCG, ECG; however, the catechin is not restricted thereto.

Methods for extracting effective components of the present invention is not restricted, and any methods known in this field can be applied without restriction.

The composition of the present invention comprising theanine and each or mixture of caffeine, genistein, L-carnitine and catechin has excellent effects of decomposing neutral fats in fat cells. More specifically, the composition of the present invention accelerates decomposition of fats by hydrolyzing triglyceride in adipocyte (fat cell) to a free fatty acid and a glycerol. This is because the composition of the present invention improves the expression of β₃-adrenergic receptor in a 3T3-L1 cell differentiated to a adipocyte to enhance and maintain the hydrolysis of triglyceride and has a function to improve the expression of enzymes related to the decomposition or combustion of fats.

Therefore, when the composition of the present invention comprising theanine and at least one selected from the group consisting of caffeine, genistein, L-carnitine and catechin is applied to the skin, the composition can help making elastic firm and smooth body figure by selectively removing subcutaneous fats.

In addition, the composition of the present invention has excellent effects on inhibiting differentiation of fat cells (adipocyte) and accumulation of fats in fat cells. That is, the composition of the present invention can inhibit enlargement of the sizes and the number of fat cells. This is because the composition of the present invention can reduce the activity of GPDH (glycerol-3-phosphate dehydrogenase) enzyme, a label component of differentiation of a fat cell, in a 3T3-L1 cell differentiated to a fat cell. Therefore, when the composition of the present invention comprising theanine and at least one selected from the group consisting of caffeine, genistein, L-carnitine and catechin is applied to the skin, increase of body fat can be effectively inhibited by the inhibition of the generation and the enlargement of fat cells.

In addition to reducing fats, the composition of the present invention can be used for external composition for anti-cellulite curing rough skin to be elastic firm and smooth by applying the composition onto a cellulite site generated due to the enlarged fat cells. In particular, because the components of the composition are extracted from green tea and soybean, it does not have undesirable side effects or harms to the skin.

In addition to the effects of decomposing and removing fats excessively accumulated in a mature fat cell, the composition of the present invention helps combustion of free fatty acid to reduce and prevent obesity. Contrary to the conventional methods or compositions that are focused on only one feature, for example on differentiation of fat cell or on acceleration of decomposition of fats, the composition of the present invention can decompose fats in fat cells already generated and can remove the by-products of the decomposition completely to prevent re-accumulation of fats, which provides novel and direct method and composition for preventing obesity.

Any conventional method can be applied to mix the components of the present invention, theanine, caffeine, genistein, L-carnitine and catechin, and one skilled in the art may modify the methods easily. In addition, additives, for example, to make the mixing easier can be adopted without restriction.

In the present invention, the amount of theanine and at least one selected from the group consisting of caffeine, genistein, L-carnitine and catechin is preferably 0.0001˜20 wt % to the total weight of the composition, but not restricted thereto.

The external application of the present invention comprises theanine and at least one selected from the group consisting of caffeine, genistein, L-carnitine and catechin, therefore, it can reduce subcutaneous fat making body figure slim and has the effects of slimming body, removing cellulite and firming body figure by the decomposition of subcutaneous fat when applied onto the skin.

The formulations for the external application of the present composition comprising theanine and at least one selected from the group consisting of caffeine, genistein, L-carnitine and catechin are not restricted on the condition that the composition is used for decomposition and combustion of fats, elasticity and firmness of skin. For example, the formulation comprises skin softener, nutrition water, nutrition lotion, massage cream, nutrition cream, pack, gel, skin adhesive type formulations, in addition to lotion, ointment, gel, cream, patch, spray or the like.

In the above skin application formulations, in addition to the effective components of theanine, caffeine, genistein, L-carnitine and catechin, any other proper components may be selected and added by one skilled in the art. By the addition of proper components, synergic effect can be accomplished.

### BRIEF DESCRIPTION OF DRAWINGS

FIG. 1 is a graph showing the effect of theanine, a component from green tea, decomposing neutral fat (triglyceride).

FIG. 1 is a graph showing the effect of theanine, a component from green tea, controlling the expression of β₃-adrenergic.

A: Control

B: Treated with 0.005% of caffeine

C: Treated with 0.005% theanine

FIG. 3 is a graph showing the effect of catechin, a component from green tea, prohibiting the differentiation of fat cell.

FIG. 4 is a graph showing the result of an experiment for synergic effects of theanine and catechin inhibiting accumulation of neutral fats in fat cells.

FIG. 5 is a graph showing the result of an experiment for synergic effects of theanine, caffeine, genistein and L-carnitine improving decomposition of fats.

FIG. 6 is a graph showing the decreasing rate of subcutaneous fat by the slimming composition of the present invention.

FIG. 7 is a graph showing the improvement of skin firmness after using the slimming composition of the present invention comprising theanine, caffeine, genistein, L-carnitine and catechin, observed with naked eyes.

### PREFERRED EMBODIMENT OF THE INVENTION

Hereinafter, the present invention is described in more detail with Examples and Experimental Examples, however the scope of the invention is not restricted thereto. The Examples and Experimental Examples are exemplified to describe the invention and it is clear to those skilled in the art that the scope of the present invention is not restricted to the Examples or Experimental Examples.

Theanine used in the following Examples and Experimental Examples is purchased from Taiyokagaku (Japan) and caffeine, genistein and L-carnitine are purchased from Sigma (U.S.A.).

### PREPARATION EXAMPLE 1

### Extraction of Catechin

2 kg of green tea leaves were soaked in 10 l of water at 80° C. for 5 hours to obtain extract and the solution was taken, in addition, the residue was also soaked in 5 l of water at 80° C. for 3 and the solution was again taken and added. The solution was filtered with filtration paper and treated with ethyl acetate to obtain ethyl acetate fraction, and treated with chloroform to remove caffeine then concentrated. The resultant solution was passed through Sepharose column and extracted with a mixture of methylene chloride and methanol (1:1), then the extracted solution was concentrated at 40° C. to obtain catechin powder.

### REFERENCE EXAMPLE 1

### Isolation of Fat Cell (Adipocyte)

Epididymal adipose tissues obtained from male SD rat were cut to small pieces, and 0.1% of collagenase (in DMEM without phenol red) was added then cultured for 2 hours at 37° C., and then filtered to obtain adipocyte (fat cell).

Then, in order to verify the ability of each component accelerating the decomposition of neutral fat in adipocytes of male SD rat, experiment was performed using the adipocytes obtained above. 1×10⁶ cells/well were cultured in DMEM (Dulbeco's modified eagles medium) containing 0.5% of bovine serum albumin (BSA) free from fatty acid for 2 hours and used in each experiment.

### REFERENCE EXAMPLE 2

### Differentiation of Fat Cell (Adipocyte)

3T3-L1 cell, fibroblast cell line of rat, was inoculated in 6 well culture plate with 1×10⁵cells/well and cultured in DMEM (Dulbecos modified eagles medium, GIBCO BRL, Life Technologes) containing 10% of fetal bovine serum (FBS). After 2 days of culture, culture medium was changed with a new DMEM (containing 10% FBS), and cultured further 2 days. Then the culture cell was deposited in a new DMEM (containing 10% FBS) containing 1 μg/ml of insulin, 0.5 mM of IBMX and 0.25 μM of dexamethasone to induce differentiation, after 2 days, the culture medium was changed with a new DMEM containing insulin, and cultured for 5 days. After 5 days of culture, the culture medium was changed with a normal DMEM (containing 10% FBS), and observed until cells changed to fat cells (adipocytes).

### REFERENCE EXAMPLE 3

### Method for Measuring Decomposition of Fat

The degree of the decomposition of fat was observed by measuring the concentration of glycerol isolated into the culture medium from fat cell. Measurement of the quantity of the glycerol was performed with chromphoric reaction method using GPO-trinder kit purchased from Sigma (St. Louis, Mo., U.S.A.), and absorption was measured in 540 nm using ELISA reader, then the result of each component was calculated based on the data of the control settled to be 100%. Control was cultured without experimental or comparative material, on the other hand, 10 μM of each effective component, theanine, caffeine, genistein, L-carnitine and catechin were added to the samples.

### EXPERIMENTAL EXAMPLE 1

### Effect of Theanine Accelerating Decomposition of Fat

In order to measure the effect of the theanine decomposing neutral fats of fat cells, 3T3-L1 fat cells differentiated in the Reference Example 2 were used.

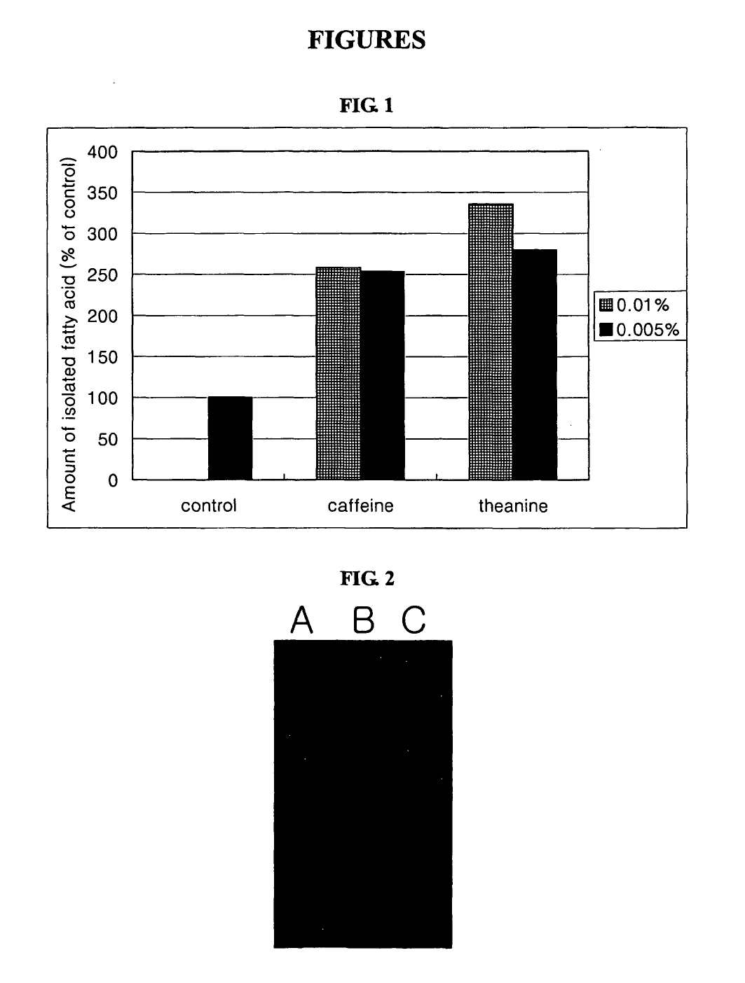

3T3-L1 fat cells were washed with PBS (phosphate buffered saline) twice, and DMEM containing 0.5% of bovine serum albumin (BSA) free from fatty acid was added thereto. Theanine was purchased from KuridaKogyo (Japan) (more than 97%), and measurement of the quantity of the glycerol was performed with chromphoric reaction method using GPO-trinder kit purchased from Sigma (St. Louis, Mo., U.S.A.), and absorption was measured in 540 nm using ELISA reader. Control was cultured without experimental or comparative material and the result of each component was calculated based on the data of the control settled to be 100%. In addition, a sample treated with same concentration of caffeine was used as a positive comparative, and the degree of decomposition of fat was observed by measuring the concentration of glycerol isolated into the culture medium from fat cell. The results are shown in FIG. 1.

As can be seen in the FIG. 1, compared with the control, the concentration of glycerol isolated into the culture medium from fat cell increased in the sample treated with theanine extracted from green tea. In addition, theanine does not cause cell toxicity at high concentration and showed more excellent effect of decomposition of fat than that of caffeine, a positive comparative control, known to have the effect of decomposing fat.

### EXPERIMENTAL EXAMPLE 2

### Effect of Theanine Controlling Expression of the β₃-Adrenergic Receptor

In order to clarify the mechanism of acceleration of decomposition of neutral fat by the theanine, 3T3-L1 fat cells differentiated in the Reference Example 2 were used. The cells were treated with 0.005% of theanine purchased from KuridaKogyo (Japan) (more than 97%) and comparative materials (caffeine, control), and 24 hours later, RNAs were extracted from the cells and performed RT-PCR. The results are shown in FIG. 2. The RT-PCR kit was purchased from TaKaRa, and primer of β₃ -adrenergic receptor was purchased from Bioneer.

As can be seen in FIG. 2, the expression of β₃-adrenergic receptor, a signal of decomposition of fat in a fat cell, increases by the treatment of theanine, and therefore it was verified that the effect of theanine decomposing fat is due to the increase of the expression of the receptor.

### EXPERIMENTAL EXAMPLE 3

### Effect of Catechin Inhibiting Differentiation of Fat Cell

In order to measure the effect of catechin accelerating the composition of neutral fat in a fat cell, an experiment of culturing and measuring absorption using fibroblast cell line was performed.

3T3-L1 fat cells differentiated in the Reference Example 2 were washed with PBS (phosphate buffered saline) three times, and harvested with extraction buffer (20 mM of Tris, 1 mM of EDTA and 1 mM of 2-mercaptoethanol). The harvested cells passed G26 needle 6 times on an ice base, centrifuged at 15000×g, 4° C. for 3 minutes and the supernatant was gathered to obtain cell extracts.

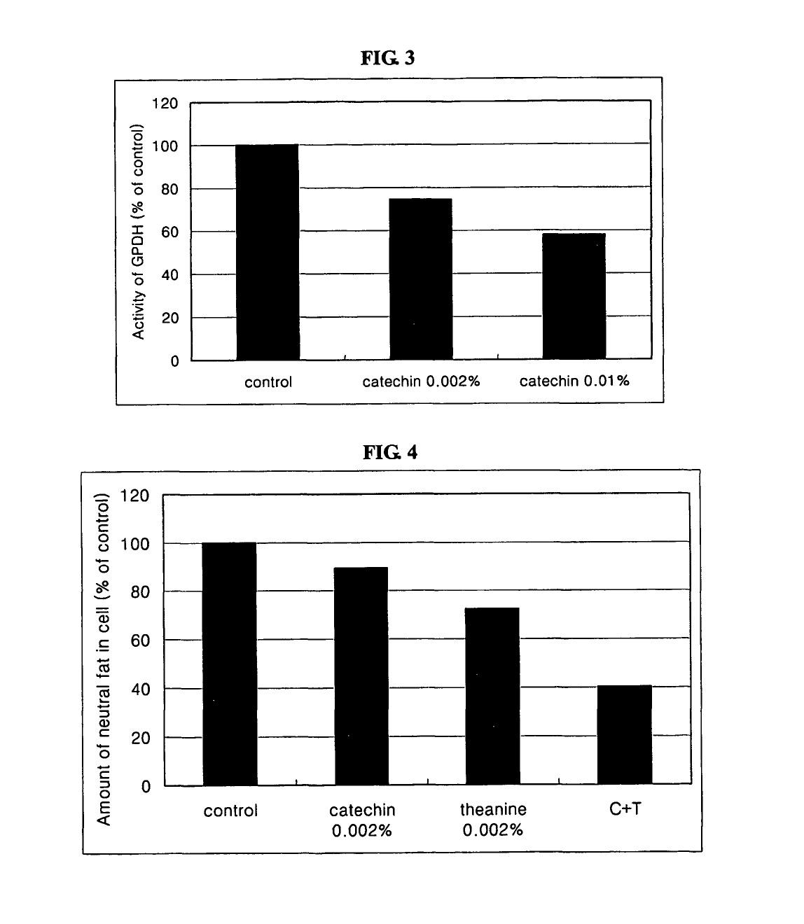

In order to measure the expression of GPDH (glycerol-3-phosphate dehydrogenase), GPDH assay buffer containing 0.1M of triethanolamine, 2.5 mM of EDTA, 0.1 mM of 2-mercaptoethanol, 125 uM of NADH (nicotinamide adenine dinucleotide, reduced form) and 100 uM of DHAP (dehydroxyacetonephosphate) was added to the above-obtained cell extracts, then the decrease of absorption was measured at 340 nm for 2 minutes. The amount of change was described with a unit of dA/min per mg. Control was cultured without experimental or comparative material and the result of each component was calculated based on the data of the control settled to be 100%. The results are shown in FIG. 3.

As shown in FIG. 3, when catechin was added to 3T3-L1 cells during cell differentiation, differentiation was significantly inhibited compared with that of control.

### EXPERIMENTAL EXAMPLE 4

### Effects of Theanine and Catechin Inhibiting Neutral Fat

In order to measure the effects of theanine and catechin affecting the generation of neutral fat, an experiment of culturing and measuring absorption using fibroblast cell line was performed.

Neutral fats in the cell extracts obtained same as Experimental Example 3 underwent chromphoric reaction using GPO-trinder kit purchased from Sigma (St. Louis, Mo., U.S.A.), and absorption was measured at 540 nm using ELISA reader. The results are shown in FIG. 4.

As can be seen in FIG. 4, compared with control, when 3T3-L1 cells were treated with theanine and catechin during cell differentiation, the amount of neutral fat decreased, and when the above two components were used together the decrease of accumulated neutral fat was more significant.

### EXPERIMENTAL EXAMPLE 5

### Synergic Effect of Theanine, Caffeine, Genistein and L-Carnitine Decomposing Fats

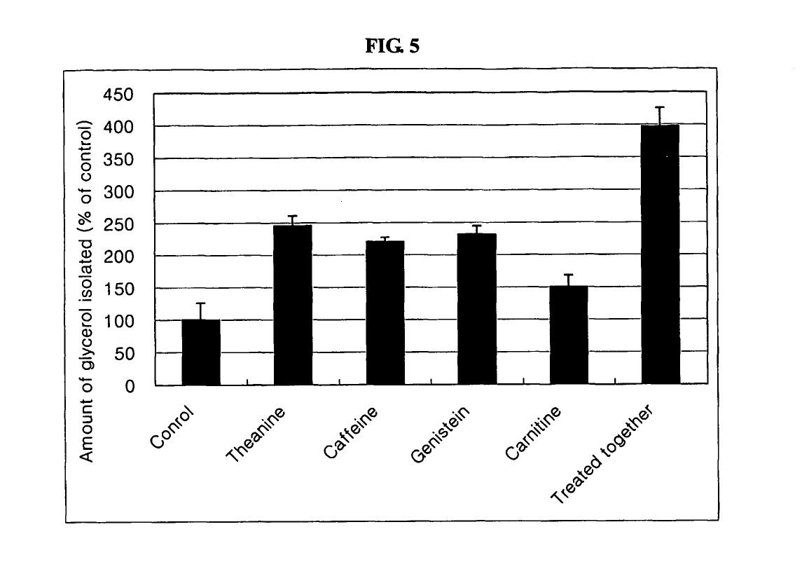

In order to verify whether theanine, caffeine, genistein and L-carnitine, each having different mechanism in the decomposition of fats, show synergic effects in the decomposition of fats or not, fat cells of SD rat isolated by the method of Reference Example 1 were treated with each of and mixture of the above materials. The concentration of each theanine, caffeine, genistein and L-carnitine was 40 μM when treated alone, and when treated together the concentration of each component was 10 μM considering concentration balance. The results are shown in FIG. 5.

As can be seen in FIG, 5, when the four components are treated together the amount of isolated glycerol increases significantly compared with when treated alone. Considering the decomposition results of fat in the Experimental Examples 14, the increase obtained in FIG. 5 is not merely because of increase of the amount of effective components but because of synergic effects by the mutual operations of each mechanism of the components.

### EXPERIMENTAL EXAMPLE 6

### Measurement of Skin Irritation of Theanine, Caffeine, Genistein, L-Carnitine and Catechin

Skin irritations caused by theanine, caffeine, genistein, L-carnitine and catechin were measured observing edema and erythema on the skin of a New Zealand white rabbit.

Vehicle, 10% of theanine, caffeine, genistein, L-carnitine, catechin or mixture thereof were applied on the skin of a New Zealand white rabbit, twice a day for 4 days, total 8 times. After application, total skin irritation index was calculated by summing up the scores of erythema and edema. The index of skin irritation was measured according to Table 1, and the results are shown in Table 2. The skin irritation index was calculated according to Draize's skin Primary Irritation Index (P.I.I.) (Draize, J. H., Appraisal of the safety of chemical in foods, drugs and cosmetics).

TABLE 1

| Degree of skin irritation | | score |
|---|---|---|
| Erythema | No erythema | 0 |
| and Crab | Very weak erythema (slightly | 1 |
| | observed in naked eyes) | |
| | Clear erythema | 2 |
| | Severe erythema | 3 |
| | Dark red strong erythema and | 4 |
| | generation of crab | |
| Edema | No edema | 0 |
| | Very weak edema (slightly | 1 |
| | observed in naked eyes) | |
| | Clear edema (distinguishable) | 2 |
| | Severe edema (swollen about 1 mm) | 3 |
| | Strong edema (swollen more than | 4 |
| | 1 mm and extended outward) | |

(Note)
Primary Irritation Index = (mean sum of score of erythema and score of dedma)/4

TABLE 2

| | Primary Irritation Index |
|---|---|
| Component | (0˜4) |
| Vehicle | 0.6 |
| 10% Solution of theanine | 0.8 |
| 10% Solution of caffeine | 0.7 |
| 10% Solution of genistein | 0.8 |
| 10% Solution of L-carnitine | 0.7 |
| 10% Solution of catechin | 0.8 |
| 10% Solution of theanine, caffeine, | 0.9 |
| genistein, L-carnitine | |
| 10% Solution of theanine, caffeine, | 0.8 |
| genistein, L-carnitine, catechin | |

As can be seen in table 2, theanine, caffeine, genistein, L-carnitine and catechin do not cause skin irritation compared with control.

### EXAMPLES 1˜8 and COMPARATIVE EXAMPLES 1˜2

Slimming/anti-cellulite lotions were prepared according to the following Table 3 and 4, which are Examples 1˜8 and Comparative Examples 1˜2.

TABLE 3

| | Example (wt %) | | | | | | | |
|---|---|---|---|---|---|---|---|---|
| Component | 1 | 2 | 3 | 4 | 5 | 6 | 7 | 8 |
| Distilled water | To | To | To | To | To | To | To | To |
| | 100 | 100 | 100 | 100 | 100 | 100 | 100 | 100 |
| Theanine | 1.0 | 1.0 | 1.0 | 1.0 | 1.0 | 1.0 | 1.0 | 1.0 |
| Caffeine | 1.0 | 1.0 | — | — | — | 1.0 | 1.0 | — |
| Genistein | 1.0 | — | 1.0 | — | — | 1.0 | — | 1.0 |
| L-carnitine | 1.0 | — | — | 1.0 | — | — | 1.0 | 1.0 |
| Catechin | 1.0 | — | — | — | 1.0 | — | — | — |
| Vegetable hydrogenated oil | 1.5 | 1.5 | 1.5 | 1.5 | 1.5 | 1.5 | 1.5 | 1.5 |
| Stearic acid | 0.6 | 0.6 | 0.6 | 0.6 | 0.6 | 0.6 | 0.6 | 0.6 |
| polyglycerol-10 pentastearic & | 1.0 | 1.0 | 1.0 | 1.0 | 1.0 | 1.0 | 1.0 | 1.0 |
| behenyl alcohol & sodium stearoyl | | | | | | | | |
| lactylate | | | | | | | | |
| Arachidyl behenyl acohol & | 1.0 | 1.0 | 1.0 | 1.0 | 1.0 | 1.0 | 1.0 | 1.0 |
| arachidyl glucoside | | | | | | | | |
| Cetylaryl alcohol & cetylaryl | 2.0 | 2.0 | 2.0 | 2.0 | 2.0 | 2.0 | 2.0 | 2.0 |
| glucoside | | | | | | | | |
| PEG-100 stearate & glycerol oleate & | 1.5 | 1.5 | 1.5 | 1.5 | 1.5 | 1.5 | 1.5 | 1.5 |
| propyleneglycol | | | | | | | | |
| caprylic/capric triglyceride | 4.0 | 4.0 | 4.0 | 4.0 | 4.0 | 4.0 | 4.0 | 4.0 |
| Meadowfoam seed oil | 3.0 | 3.0 | 3.0 | 3.0 | 3.0 | 3.0 | 3.0 | 3.0 |
| Cetyl octanoate | 4.0 | 4.0 | 4.0 | 4.0 | 4.0 | 4.0 | 4.0 | 4.0 |
| cyclomethycon | 6.0 | 6.0 | 6.0 | 6.0 | 6.0 | 6.0 | 6.0 | 6.0 |
| Methyl paraben | 0.2 | 0.2 | 0.2 | 0.2 | 0.2 | 0.2 | 0.2 | 0.2 |
| Propyl paraben | 0.1 | 0.1 | 0.1 | 0.1 | 0.1 | 0.1 | 0.1 | 0.1 |
| Sodium EDTA | 0.02 | 0.02 | 0.02 | 0.02 | 0.02 | 0.02 | 0.02 | 0.02 |
| Triethanol amine | 0.13 | 0.13 | 0.13 | 0.13 | 0.13 | 0.13 | 0.13 | 0.13 |
| Glycerine | 8.0 | 8.0 | 8.0 | 8.0 | 8.0 | 8.0 | 8.0 | 8.0 |

TABLE 4

Comparative Example (wt %)

| Component | 1 | 2 |
|---|---|---|
| Distilled water | To 100 | To 100 |
| Theanine | — | 1.0 |
| Caffeine | — | — |
| Genistein | — | — |
| L-carnitine | — | — |
| Catechin | — | — |
| Vegetable hydrogenated oil | 1.5 | 1.5 |
| Stearic acid | 0.6 | 0.6 |
| polyglycerol-10 pentastearic & | 1.0 | 1.0 |
| behenyl alcohol & sodium | | |
| stearoyl lactylate | | |
| Arachidyl behenyl acohol & | 1.0 | 1.0 |
| arachidyl glucoside | | |
| Cetylaryl alcohol & cetylaryl | 2.0 | 2.0 |
| glucoside | | |
| PEG-100 stearate & glycerol | 1.5 | 1.5 |
| oleate & propyleneglycol | | |
| caprylic/capric triglyceride | 4.0 | 4.0 |
| Meadowfoam seed oil | 3.0 | 3.0 |
| Cetyl octanoate | 4.0 | 4.0 |
| cyclomethycon | 6.0 | 6.0 |
| Methyl paraben | 0.2 | 0.2 |
| Propyl paraben | 0.1 | 0.1 |
| Sodium EDTA | 0.02 | 0.02 |
| Triethanol amine | 0.13 | 0.13 |
| Glycerine | 8.0 | 8.0 |

### EXAMPLE 7

### Slimming Effect of the Composition

In order to verify the slimming effects of the compositions prepared in the Examples 1˜8 and Comparative Examples 1˜2, 100 of adult females (10 females for each group) of 20˜30 years old who do not have any problems in metabolism and whose BMI [Body Mass Index, (weight(kg)/height(m))²) is ranged from 23 to 25 were selected. Then the lotion compositions prepared in the Examples 1˜8 and Comparative Examples 1˜2 were applied to them for 8 weeks twice a day on the inner part of thighs with massaging. Thicknesses of the subcutaneous fats were measured before and after 8 weeks of using the lotion to verify the effects.

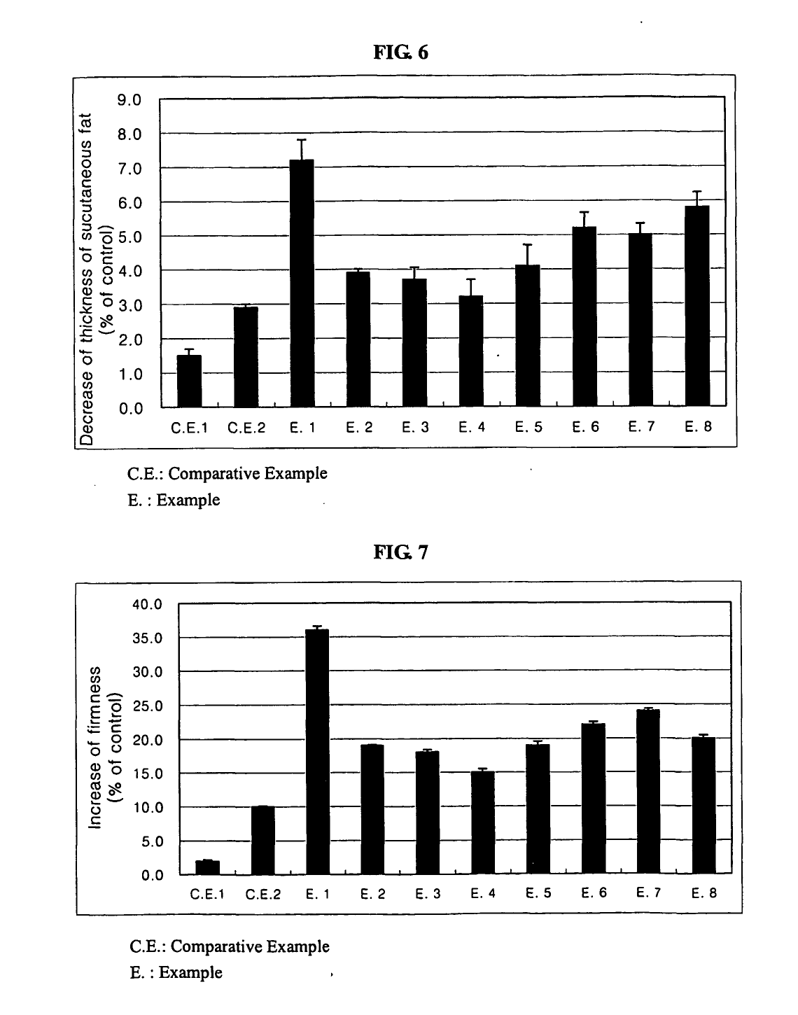

Measurement of the thickness of the subcutaneous fat was performed using Ultrasound-EuB 415 US scanner with ultrasonic waves (unit: mm), data was treated by a method of Student t test as a positive verification, and the results before and after using the compositions were compared analyzing statistic significance (significance p<0.05). The results are shown in FIG. 6.

As can be seen in FIG. 6, Examples 1˜8 comprising effective components showed more significant effects of decreasing the thickness of subcutaneous fat than that of Comparative Example 1 not containing effective component, and that of Comparative Example 2 containing only theanine. In particular, Example 1 containing all the 5 effective components showed the most significant effect.

### EXPERIMENTAL EXAMPLE 8

### Firming Effect of the Composition

In order to verify the firming effects of the compositions prepared in the Examples 1˜8 and Comparative Examples 1˜2, skin firmness was observed with naked eyes. The skin firmness were scored by the examiner from 1 to 9, and the scores before and after using the compositions were compared by analyzing statistic significance with Wilcoxon test (significance α=0.05). The results are shown in FIG. 7.

As can be seen in FIG. 7, Example 1 containing the effective components showed the increased skin firmness.

As shown in the above, the composition of the present invention comprises theanine, caffeine, genistein, L-carnitine and catechin and has excellent effects of decomposing neutral fats in fat cells, and accelerates decomposition of fats by hydrolyzing triglyceride in adipocyte (fat cell) into free fatty acid and glycerol, and can prohibit obesity and reduce cellulite to make the more firm and smooth skin and body figure. Therefore, the composition of the present invention can be is applied to the cosmetic compositions for slimming and anti-cellulite, which can develop cosmetic industry.

The present invention was described with reference to examples, however a person skilled in the art can modify the invention within the scope of the present invetnion, and it is clear that the modified invention would be in the scope of the present invention.

1. A composition for slimming comprising theanine and at least one selected from the group consisting of caffeine, genistein, L-carnitine and catechin as effective components, which accelerates decomposition of fats in fat cells.

2. The composition according to claim 1, said theanine is at least one selected from the group consisting of L-theanine, D-theanine and DL-theanine.

3. The composition according to claim 1, said catechin is at least one selected from the group consisting of (+) catechin (C), (−) epicatechin (EC), (−) epigallocatechin-3-gallate (EGCG), (+) epigallocatechin (EGC) and (−) epicatechin gallate (ECG).

4. The composition according to claim 1, said theanine improves the expression of β₃-adrenergic receptor to accelerate decomposition of fats.

5. The composition according to claim 1, the components are contained in an amount of 0.0001˜20 wt % to the total amount of the composition.
